(19) United States
(12) Patent Application Publication
(10) Pub. No.: US 2020/0405264 A1
(43) Pub. Date: Dec. 31, 2020

(54) REGION OF INTEREST POSITIONING FOR LONGITUDINAL MONTIORING IN QUANTITATIVE ULTRASOUND

(71) Applicant: Siemens Medical Solutions USA, Inc., Malvern, PA (US)

(72) Inventors: Yassin Labyed, Maple Valley, CA (US); John Benson, Issaquah, WA (US)

(21) Appl. No.: 16/454,855

(22) Filed: Jun. 27, 2019

(51) Int. Cl. A61B 8/00; A61B 8/08; G06T 7/00

(52) U.S. Cl. CPC A61B 8/469; A61B 8/485; G16H 30/40; G06T 7/0012; A61B 8/461; A61B 8/5215

(57) ABSTRACT

For longitudinal monitoring of a patient using quantitative ultrasound (QUS), one or more indicators of region of interest (ROI) position relative to the patient and one or more images from a past QUS imaging for the patient are stored. The indicator is in addition to an image with the ROI. For subsequent QUS imaging of the patient, the indicator is used to position the ROI. In QUS monitoring, a same or fixed anatomy is monitored from examination-to-examination based on placement of the ROI using a displayed indicator for the previous placement.

### BACKGROUND

The present invention relates to quantitative ultrasound imaging. In quantitative ultrasound (QUS) imaging, the detected information is further processed to quantify a biomarker or characteristic of the tissue being imaged. Rather than merely providing a B-mode image of the tissue, a characteristic of that tissue is imaged. For example, shear wave speed in the tissue is calculated using ultrasound imaging. Other examples include strain, attenuation, or backscatter measures.

For quantitative ultrasound imaging, a user typically positions a region of interest (ROI) in a B-mode image. To avoid delays or processing complications for quantification over the entire field of view (FOV) of the B-mode image, the user-positioned ROI defines the region of tissue for quantification.

QUS biomarkers hold promise not only for screening and diagnosis, but also for monitoring disease progression or response to treatments from lifestyle, dietary, and/or pharmaceutical interventions. Since different QUS examinations are performed before and after treatment, the monitoring is sensitive to proper placement of the FOV and ROI in the different examinations. To monitor changes in tissue properties of a fixed anatomical location, sonographer experience is relied on to find the same FOV and ROI. The matching of ROIs over time or in different examinations may be subjective and inaccurate. As a result, the comparison of QUS biomarkers is less diagnostically or prognostically reliable.

### SUMMARY

By way of introduction, the preferred embodiments described below include methods, computer readable storage media, instructions, and systems for ROI positioning in QUS imaging. For longitudinal monitoring using QUS, one or more indicators of ROI position relative to the patient and one or more images from a past QUS imaging for the patient are stored. The indicator is in addition to an image with the ROI. For a subsequent QUS imaging of the patient, the indicator is used to position the ROI. In QUS monitoring, a same or fixed anatomy is monitored from examination-to-examination based on placement of the ROI using a displayed indicator for the previous placement.

In a first aspect, a method is provided for ROI positioning in QUS imaging with an ultrasound scanner. An indicator of the ROI for first anatomy of a patient from a previous quantitative ultrasound examination is displayed. Another quantitative ultrasound examination is performed with the ROI for this other quantitative ultrasound examination positioned on the first anatomy of the patient based, in part, on the indicator.

In a second aspect, a method is provided for ROI positioning in QUS imaging with an ultrasound scanner. A quantitative ultrasound image with a region of interest is stored. Input of a relative position of a transducer probe to the patient is received. The relative position is for when the quantitative ultrasound image is generated. The relative position is stored linked with the quantitative ultrasound image.

In a third aspect, a system is provided for ROI positioning in QUS imaging. An image processor is configured to assist positioning of the ROI over a same anatomy of a patient used in a previous instance of quantitative ultrasound imaging. A display is configured to display a current instance of quantitative ultrasound imaging of the same anatomy based on the position of the ROI.

The present invention is defined by the following claims, and nothing in this section should be taken as limitations on those claims. Further aspects and advantages of the invention are disclosed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a flow chart diagram of one embodiment of a method for ROI positioning by receiving and storing an indicator of ROI position;

FIG. 2 is an example QUS image with a positioned ROI and a pictogram as an indicator;

FIG. 3 is an example indicator based on a body model;

FIG. 4 is a flow chart diagram of one embodiment of a method for ROI positioning using an indicator from a previous examination; and

FIG. 5 is a block diagram of one embodiment of a system for ROI positioning in QUS imaging.

### DETAILED DESCRIPTION OF THE DRAWINGS AND PRESENTLY PREFERRED EMBODIMENTS

Automatic ROI placement is provided in shear wave or other quantitative imaging. An indicator of ROI placement from a previous examination is used to assist in placement of the ROI in a current examination, providing QUS measurements of the same anatomy in different examinations. The indicator is in addition to or different than showing the ROI on an image. The indicator assists in positioning the FOV and the ROI. ROI positioning is provided for longitudinal monitoring using QUS. For ultrasound to be useful for monitoring, the process for finding and measuring the same anatomical area is automated.

In one embodiment, an indicator based on similarity measures between current and reference views are used for ROI positioning. The process of finding the same anatomical location is automated using indicators and correlation techniques.

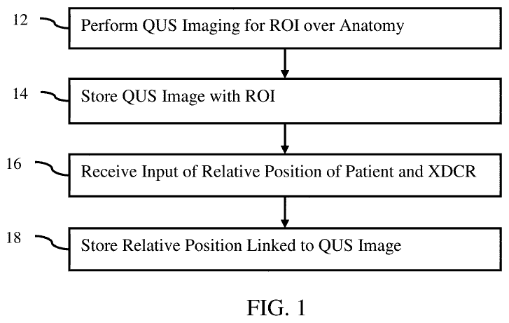

FIG. 1 shows one embodiment of a method for ROI positioning in quantitative ultrasound imaging with an ultrasound scanner. For longitudinal study, the same anatomy is imaged with QUS imaging at different times or for examinations. The examinations may be separated by a treatment and/or hour or more. The examinations may be performed by the same or different sonographers. The quantification results are more useful if for the same anatomy, allowing comparison or study of change. The subsequent examination should perform QUS imaging for the same ROI relative to the patient anatomy.

FIG. 1 is directed to the initial or earlier QUS imaging. One or more indicators are created to assist in ROI positioning for subsequent QUS imaging of the patient. The created indicators assist in monitoring a fixed anatomical location over time using QUS.

The method is performed by the system shown in FIG. 5 or a different system. For example, a medical diagnostic ultrasound imaging system performs QUS imaging, stores a QUS image with an ROI, solicits and receives input of an indicator for that QUS image, and stores the indicator linked to the QUS image. Other devices may perform any of the acts, such as a picture archiving and communications system (PACS) or computerized medical records database storing the indicator and QUS image.

The acts are performed in the order shown or another order. For example, act 14 is performed at a same time as act 18 or after act 16. As another example, act 16 with or without act 18 is performed as part of act 12 or even upon placement of an ROI before act 12.

Additional, different or fewer acts may be used. For example, acts for configuring the ultrasound scanner to perform QUS imaging are included. As another example, acts for the review or other use of the QUS image are performed.

In act 12, a medical diagnostic ultrasound imaging system or scanner performs QUS for a patient. The QUS may be limited to a ROI in a field of view (FOV) of the scanner or transducer.

To locate the ROI for quantitative imaging, ultrasound data representing or responsive to a patient is acquired. An ultrasound imaging system or scanner scans the patient. Alternatively, the data is acquired from a previous scan by the scanner, such as by transfer from a memory or picture archiving and communications system.

This scan is an initial scan, such as a first scan or a later scan once quantitative imaging is to be used. For example, the scanning is repeated as a sonographer positions the transducer to scan the desired region of the patient. The FOV for the scanning is positioned over the organ or organs of interest. Once the object of interest is in the FOV, the ultrasound data to be used for locating the ROI is available from the scanning or is acquired by further scanning.

The scan for ultrasound data to locate the ROI is of the entire FOV. In addition to position and orientation of the transducer, the lateral or azimuth extent and depth of the scanning define the FOV. Based on different settings, different sizes of FOV may be provided. The user or the system determines the FOV.

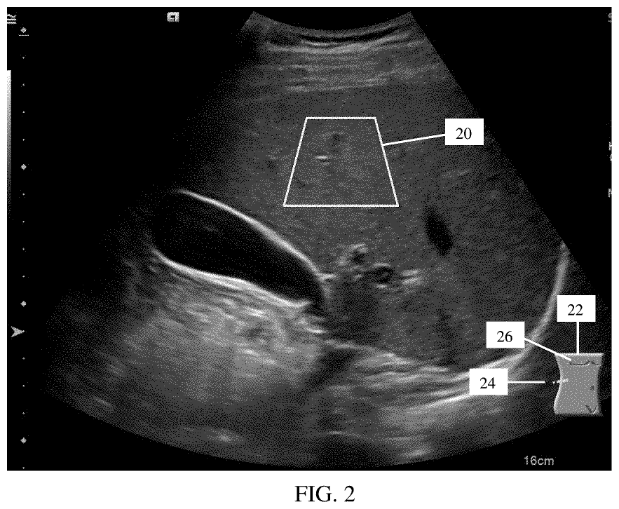

A two-dimensional image may be generated. B-mode frames of data are generated by B-mode scanning. A B-mode image represents the intensity or strength of return of acoustic echoes in the B-mode FOV. FIG. 2 shows an example B-mode image for a liver of a patient. The intensities or B-mode data are mapped to gray scale within the dynamic range of the display. In other embodiments, other types of detection and corresponding scans are performed. For example, color flow (e.g., Doppler) estimation is used. Velocity, power, and/or variance are estimated. As another example, harmonic mode is used, such as imaging at a second harmonic of a fundamental transmit frequency. Combinations of modes may be used.

The initial scan or scans of the FOV are performed prior to separate scans of the ROI 20 for quantitative imaging. The scanning is configured to cease scans of the FOV of the patient while scanning the ROI 20 for quantification. Alternatively, B-mode imaging and quantitative imaging are interleaved.

The ROI 20 is positioned in the FOV for QUS. The user, using a user interface, may position the ROI on a B-mode or other ultrasound image. Alternatively, the ultrasound scanner, such as using an image processor or controller, determines a position of an ROI 20 in the FOV of the ultrasound image. In one embodiment, a machine-learnt network is applied. The machine-learnt network associates input features, such as the ultrasound image, landmark locations, clutter levels by location, and/or fluid locations to place of the ROI 20. The application of the machine-learnt network outputs a position for the ROI 20. In an alternative embodiment, the determination uses rules. For example, the ROI 20 positioned relative to but spaced away from a landmark while also avoiding clutter and fluid. The rules may indicate a specific orientation and distance from the landmark with tolerances for orientation and distance to account for avoiding clutter and fluid. Fuzzy logic may be used.

The ROI 20 may be positioned based on the type of QUS to be performed. For shear wave speed imaging of the liver, the ROI 20 is positioned relative to a liver capsule. The ROI 20 may be positioned based on the liver capsule and to avoid fluid and relatively higher clutter.

The ROI 20 is a scan region within the FOV. The ROI is shaped based on the scan line distribution. For linear scans, the scan lines are parallel. The resulting ROI is a square or rectangular box. For sector or Vector scans, the scan lines diverge from a point on the transducer face or a virtual point positioned behind the transducer, respectively. The sector and Vector scan formats of scan lines scan in a fan shaped ROI 20. The Vector scan may be a fan shaped region without the origin point included, such as resembling a trapezoid (e.g., truncated triangle) (e.g., see ROI 20 of FIG. 2). Other shapes of ROIs 20 may be used, such as square or rectangular in a sector or Vector® scan.

The orientation may also be determined to include or avoid certain locations. The orientation may be based on the limits on steering from a transducer, detected landmarks that may cause acoustic shadowing, and/or directivity response of the tissue being quantified.

The ROI 20 has a default size. The ROI 20 is any size, such as 5 mm in lateral and 10 mm in axial. The ROI 20 is sized to avoid fluid locations or relatively high clutter. Alternatively, the ROI 20 is sized to include locations of relatively higher backscatter (e.g., lower clutter and lower noise).

The quantification scan may be affected by the size of the ROI 20. For shear wave imaging and other quantification scanning, the quantification relies on repetitive scanning of the ROI 20. By sizing the ROI smaller, the speed of scanning may increase, making the quantification less susceptible to motion artifact. By sizing the ROI 20 larger, a more representative sampling for quantification may be provided. The ROI 20 is sized as appropriate for the type of quantification. Different sizes may be selected based on a priority and avoidance of locations that may contribute to inaccuracy or artifacts.

The ROI 20 defining the scan region for quantitative imaging is less than the entire FOV of the B-mode image. FIG. 2 shows the ROI 20 as less than 30%, 20%, or 10% of the area of the FOV of the B-mode image.

The ROI 20 is positioned for quantification of particular tissue or anatomy of interest. The size, shape, and orientation are set so that particular anatomy of the patient is within the ROI 20. Different anatomy or types of tissue may be included depending on the type of QUS imaging.

Once the ROI 20 is positioned, the ultrasound scanner performs the quantitative imaging. The ROI 20 or ROIs 20 define the locations of scanning for the quantitative imaging. For example, shear wave imaging is performed by the ultrasound scanner by scanning at the position of the ROI 20. Shear wave imaging may be used to quantify diagnostically useful information, such as the shear wave speed in tissue, Young's modulus, or a viscoelastic property. Shear wave imaging is a type of ARFI imaging where ARFI is used to generate the shear wave, but other sources of stress and/or other types of ARFI (e.g., elasticity) imaging may be used. Other types of quantitative imaging, such as strain, elasticity, backscatter, or attenuation, may be used.

The quantitative imaging results in a QUS image. The QUS image includes the values of the quantitative parameter or parameters for the ROI 20. For example, the shear wave velocity as a function of location in one, two, or three dimensions is included in the QUS image. Image is used to reflect ultrasound data that may be used to form a display image or ultrasound data formatted to be or that has been displayed. In another example, the QUS image includes a quantitative value for the entire ROI.

The QUS image may include other information. For example, QUS values are used for the ROI 20 and locations in the FOV outside the ROI 20 are formed from the B-mode image. In one embodiment, the QUS image includes a reference volume, such as B-mode data in three dimensions as well as a two-dimensional B-mode image with overlaid QUS information for the ROI 20 in the two-dimensional B-mode image.

The QUS image includes a graphic or defined ROI position. Alternatively, the locations of the QUS measurements indicate the position of the ROI 20. While a display of the QUS image may cover or not use B-mode data for locations within the ROI 20, the B-mode data being replaced may be provided as part of the QUS image even if not displayed.

In act 14, the QUS image with the ROI 20 is stored. The ultrasound scanner, workstation, computer, or other processor stores the QUS image with the ROI 20 in a memory, such as PACS memory, computerized medical record, or local memory (e.g., memory of the scanner).

More than one QUS image may be stored. All QUS images for a given ROI 20 may be stored. Alternatively, the user selects one or a sub-set of the QUS images from an examination for storage.

In act 16, input of a relative position of a transducer probe to the patient is received. The input is received by the ultrasound scanner, computer, workstation, or other processor. The input is from a user interface. In alternative embodiments, the input is from a processor performing data processing, such as using a position sensing system. Magnetic position sensors on the transducer and/or position sensing of the patient and transducer (e.g., a camera) may be used to input the relative position of the transducer probe to the patient.

The relative position is for when the QUS image is generated. The same relative position or different relative positions are provided for different QUS images. The relative position may be input before, during, or after generating the QUS image but represents the relative position during scanning to generate the QUS image.

The user may provide the relative position information. FIG. 2 shows one embodiment where the input is on a pictogram 22. The QUS image is displayed with the ROI 20 and the pictogram 22. In alternative embodiments, the pictogram 22 is displayed without the QUS image. The pictogram 22 represents the patient or part of the patient. FIG. 2 shows a pictogram for abdominal acquisition. Other parts of the patient may be represented. The pictogram 22 is a graphic or icon representing the body of the patient.

The pictogram 22 depicts one or more landmarks or body markers 26 of the patient, such as a belly button, crotch, and pectoral muscles in FIG. 2. Pictograms 22 representing different parts of the patient may have different combinations of body markers 26. The body markers 26 are at different positions on the pictogram 22, such as with a spatial distribution representative of the human body.

To input the relative position, the user indicates a location on the pictogram 22 of the transducer 24. For example, a mouse or other input device is used to place and activate a pointer on the pictogram 22 at the location of the transducer 24 relative to the patient.

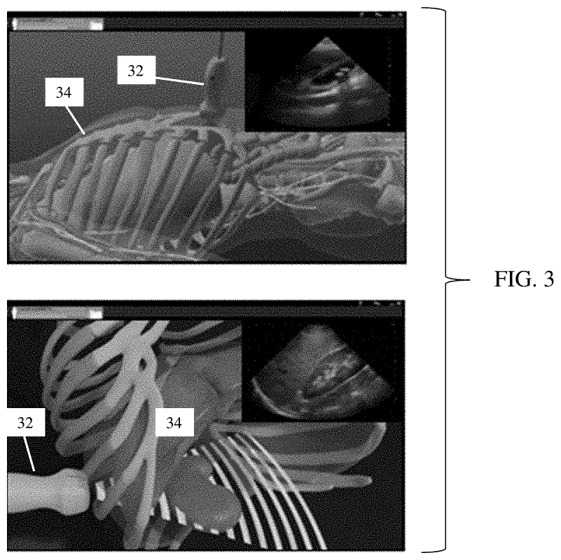

FIG. 3 shows another embodiment. Instead of a pictogram, a body model 34 is displayed. The body model 34 represents interior parts of the human body. The body model 34 may be personalized to the patient or is generic. One or more organs, bones, or other interior structure of the patient are represented. Alternatively or additionally, the body model 34 represents exterior anatomy of the patient.

The body model 34 is a three-dimensional model, representing the body in three dimensions. The body model 34 is rendered to one or more two-dimensional images from one or more different viewing directions. In alternative embodiments, one or more two-dimensional representations form the body model 34.

The input is of a location of the transducer model 32 relative to the body model 34. The location may be a relative position or point of contact. The location may include an orientation of the transducer model 32 relative to the body model 34. The user places the transducer model 32 relative to the body model 34 to represent the location of the transducer probe relative to the patient for the QUS image. The QUS image may or may not be displayed for the input.

In addition to or as an alternative to user input, a position sensor on the transducer probe and/or one or more sensors (e.g., camera) for the position of the patient and/or the transducer probe are used to provide the input. The sensed relative position may be displayed for confirmation by the user.

In act 18, the relative position is stored. The ultrasound scanner, workstation, computer, or other processor stores the relative position. The pictogram 22 with the indicated transducer 24 location or the body model 34 with the positioned transducer 52 are stored. Other parameterization may be used, such as storing the point relative to the pictogram 22 or body model 34 or storing an image of the pictogram 22 with the transducer 24 or body model 34 with the transducer 52.

The relative position is stored in a memory, such as PACS memory, computerized medical record, or local memory (e.g., memory of the scanner). More than one relative position may be stored, such as storing different relative positions for different QUS images.

The relative position, as stored, is linked with the QUS image. The relative position and QUS image may be stored together, such as in a same file. Alternatively, the link is by reference between separately stored files for the QUS image and the relative position. The link may be by storage of an image showing both the QUS image and the relative location (e.g., pictogram 22 and body model 34), such as storing the images of FIG. 2 or 3. For later access to assist in ROI positioning for a subsequent QUS examination, the QUS image, ROI position within the QUS image, and relative position of the transducer to the patient are stored.

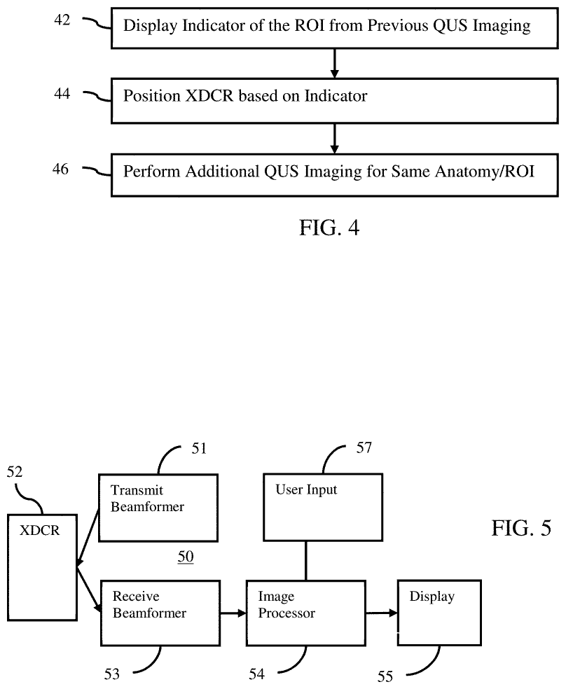

FIG. 4 shows one embodiment of a method for ROI positioning in QUS imaging with an ultrasound scanner. For longitudinal monitoring with QUS, a subsequent QUS examination is performed. The subsequent QUS examination is separated from an earlier QUS examination by one, twelve, or other number of hours and/or by treatment. Other separations may be provided where different instances of QUS imaging for the same patient occur, such as whether the ultrasound system has been powered down in between or a break of 30 minutes to allow for treatment has occurred, resulting in separate instances of QUS imaging.

In the subsequent QUS examination, the goal is to measure the same anatomy of the patient. The ROI 20 from the previous QUS examination indicates the anatomy to be measured. The goal is to perform QUS for all or a sub-set of the same locations of the patient and/or ROI 20.

The same or different ultrasound scanner and/or sonographer may be used for the different QUS examinations. An ultrasound scanner performs the acts, such as the system of FIG. 5. The user or a robotic system may perform act 44.

Additional, different, or fewer acts may be provided. For example, act 44 is not performed from the perspective of the ultrasound scanner, which scans once positioned but may not position. As another example, acts for use of the results of the QUS imaging, such as comparison of QUS results from different examinations for diagnosis, prognosis, and/or treatment planning, are provided.

The acts are performed in the order shown (e.g., top to bottom or numerical) or other orders. For example, acts 42 and 44 are interleaved or performed simultaneously.

In act 42, the ultrasound scanner displays an indicator of the ROI once QUS examination is started. The indicator indicates the ROI by showing a position of the FOV including the ROI, relative position of the transducer to the patient, and/or position of the ROI in a current FOV. The indication may be a direction or movement to provide for the current ROI to match a previous ROI or may be an indication of location and/or orientation. The indication assists in aligning a current FOV and/or ROI to match the ROI from a previous QUS examination.

The previous QUS image from the previous QUS examination is displayed to assist the user in placing the current ROI. The corresponding FOV for the ROI placement is to be found. Other images showing anatomy of interest may be displayed, such as displaying a B-mode image with a QUS overlay or separately from the QUS values.

The indicator is displayed with the image, such as side-by-side or overlaid. Alternatively, the indicator is displayed separately from the image. The display of the indicator is part of the QUS examination. By configuring the scanner for longitudinal study using QUS, the ultrasound scanner causes display of the indicator to assist in positioning the ROI over the same anatomy of the patient.

The indicator is loaded from memory or generated based on information loaded from memory. For example, the stored relative position and/or QUS images from the previous QUS examination are loaded and used to generate the indicator.

The indicator is a graphic, such as a pictogram, body model, relative position of transducer to the patient, directional arrow, similarity value, or other information for finding the FOV and/or ROI position in the FOV. Other indicators may be used.

In one embodiment, multiple indicators are provided. For example, information relating a transducer position to the patient is displayed as one indicator, and then a refinement based on a measure of similarity is displayed as another indicator. This two-step approach is used for monitoring a fixed anatomical location over time or over different examination instances using QUS. In alternative embodiments, one indicator of either the information relating relative position or refinement is used. Three or more indicators may be used. Rather than sequential display of indicators, different indicators may be displayed as a same time.

The relative position information may be a macro indicator, indicating the general area of the FOV and ROI. For example, FIG. 2 shows display of the pictogram 22 as the indicator. The location of the transducer 24 relative to the patient and corresponding one or more body markers 26 are displayed, indicating where to place the transducer relative to the patient for the current QUS examination. As another example, FIG. 3 shows display of the body model 34 with the transducer model 32 as the macro indicator. The body model 34 of internal structure of the body and the transducer model 32 position relative to the body model 34 for QUS imaging are displayed. The indicator may be displayed from different view directions or is from a single view direction of the body model 34. In other examples, other macro indicators showing relative position of the FOV or ROI to the patient may be used, such as showing a graphic of the FOV or ROI within the body model 34 or on the pictogram 22. Other representations of the patient, FOV, ROI, and/or transducer may be used.

The refinement information may be a micro indicator. The micro indicator shows information used to adjust or refine the FOV and/or ROI. For example, the macro indicator is used to generally position the transducer against the patient, and then the micro indicator is used to more precisely shift the FOV and/or ROI to match the previous FOV and/or ROI.

One example micro indicator is a display of a degree of similarity between the FOV and/or ROI as currently positioned and the FOV and/or ROI from the previous QUS examination. Where the anatomy being represented in B-mode or other images is the same, the similarity will be greater. Where the anatomy is different, the similarity will be lesser. The user uses the indication to change the FOV and/or ROI to find a sufficient or maximum degree of similarity.

In one embodiment, the ROI for QUS is automatically positioned by a processor of the ultrasound scanner. As the user performs surveillance scanning (e.g., B-mode scans while moving the transducer to find the FOV), the ROI is automatically positioned on each image. The indicator shows the degree of similarity (e.g., as a percentage, colored bar, or other indication) between the current view (i.e., current FOV or B-mode image) and the reference view (i.e., previous FOV or B-mode image). In another approach, each current image is searched for different ROI positions to find the ROI position in the current FOV with a greatest or sufficient similarity to the ROI of the previous view. As different FOVs occur during searching, the indicator is the degree of similarity for the best ROI of the current image to the ROI of the image from the previous QUS examination. The user uses the indication of similarity to position the FOV and/or ROI in the current examination.

The similarity is measured between B-mode or other modes of data. The ROI and/or FOV with a greatest similarity is based on comparison of the B-mode or other ultrasound data from one FOV and/or ROI to another FOV and/or ROI. While the ROI is for QUS, the B-mode or other data indicating anatomy, flow, and/or other distinguishing structure is used for the measure of similarity.

Any similarity measure may be used. For example, auto-correlation is used. As another example, a minimum sum of absolute differences is used. In another example, auto-correlation in conjunction with algorithms for recognizing anatomical structures is used. For example, the organ is identified. The indicator is weighted based on the organ. Where the ROI is for the liver, the organ identified as other than the liver in a FOV may provide indication of no match. Where the organ is identified as the liver, then an organ match is indicated. Further refining is provided using similarity.

As another example micro indicator or refining indictor, a direction of movement is indicated. An arrow, color, graphic, animation, or other marking indicates which direction to translate, wobble, and/or rotate the ultrasound transducer to better match the FOV and/or ROI. The refinement indicator notifies the user to move the transducer probe in a specific way and/or direction.

Where the reference image is a volume scan, the volume may be searched to fine a plane best matching a current FOV. The direction to shift the current FOV to the plane of the FOV for the ROI of the previous QUS examination is determined and indicated. Alternatively, a trend in the similarity is used to determine the direction. Where the similarity is decreasing, the indicator is to move in the opposite direction. Where the similarity is increasing, the indicator is to continue moving the current direction.

Other refinement indicators for more exactly matching the anatomy for QUS examination may be used. Any combination of micro and/or macro indicators may be used to assist in positioning the ROI. The ROI is positioned by finding the matching FOV, which includes the same anatomy as the ROI in the previous QUS examination. The assistance may also include positioning the ROI within the matching FOV, such as placing a same sized, shaped, and oriented ROI based on greatest degree of similarity. ROIs of different size, shape, and/or orientation but covering at least part of the same anatomy may be used.

In act 44, the transducer probe is positioned relative to the patient. A robotic arm or a user (e.g., sonographer) positions the transducer probe. The positioning establishes, in part, the FOV for current imaging. The transducer probe is positioned for performing QUS examination so is positioned to have a FOV that includes the same anatomy.

The positioning is based on one or more indicators. For example, the placement of the transducer probe against the skin of the patient is based on a macro indicator. The point or area of contact on the skin is derived from the macro indicator. The orientation may likewise be based on the macro indicator. Once scanning occurs, the FOV position may be refined using one or more micro indicators. The sonographer rotates, wobbles, or translates the probe based on the indicator to better match the current FOV to include the ROI from the previous QUS examination.

Once the matching FOV is identified, the ROI is manually placed to cover the same or overlapping anatomy. An indicator, such as similarity, may be used to guide placement of the ROI. Alternatively, an image processor places the ROI, based on the similarity. The indicator is displayed for the user to confirm proper placement.

In act 46, the ultrasound scanner performs another QUS examination. Based on the indicators, the FOV and/or ROI including at least some or all of the locations of the ROI in a previous QUS examination is located automatically, semi-automatically, or manually. The current ROI is placed and used for QUS measurements. For example, when a similarity measure is maximized for a current ROI relative to the past ROI, then QUS examination is begun automatically or triggered by the user. Since the current ROI is positioned on at least some of the same anatomy as the past ROI, the QUS measurements are for at least some of the same anatomy.

A QUS image is generated. The QUS image shows values for a QUS parameter. For example, shear wave velocity, attenuation, or backscatter is determined from ultrasound data. The same type of QUS examination is performed for the current examination as for a past examination. Since the ROI is for the same anatomy, at least in part, then the values of the QUS parameter or parameters may reflect changes in the anatomy due to treatment.

The generated image is displayed on a display device. The image processor, renderer, or other device generates an image from the QUS imaging for the ROI or ROIs. The image includes one or more quantities representing tissue characteristics. An alphanumeric or graphical representation of one or more quantities may be provided, such as a shear wave speed V, for the ROI overlaid as an annotation with a B-mode image. Alternatively or additionally, the quantities for different locations are displayed. For example, the quantities for different locations in the ROI modulate the brightness and/or color so that spatial representation of the quantity is provided in the image. The spatial representation may be overlaid or included in a B-mode or other image. The quantity or quantities may be provided without other types of imaging or may be added to or overlaid with other types of ultrasound imaging.

For longitudinal monitoring, the QUS images and/or values of the quantifications may be displayed adjacent to each other. The adjacent display of images for the QUS ROIs, images for the FOV with QUS ROIs, or other images may allow for subjective comparison. Alternatively or additionally, a difference is calculated. For example, location-by-location differences in the values of the QUS parameters are calculated to show change over time. An image showing spatial distribution of the change relative or not relative to surrounding anatomy is generated and displayed. Alternatively, an annotation showing an average difference or difference in ROI representative values (i.e., single QUS parameter value for the entire ROI at each time) is displayed.

In one embodiment, parameters derived from QUS measurements at different time points are calculated and displayed. For example, the percent change or ratio of shear wave speed or fat fraction before and after intervention is calculated and displayed. In other embodiments, a curve, table, or graph showing the value of a QUS over time or examinations is displayed.

The QUS ultrasound imaging is used for diagnosis, prognosis and/or treatment guidance. Enhanced, more consistent, and/or more accurate quantitative imaging due to proper ROI placement for different examinations leads to better diagnosis, prognosis, and/or treatment by a physician. The physician and patient benefit from the improvement as the output of the quantification is more likely reflective of the same anatomy.

FIG. 5 shows one embodiment of a system 50 for ROI positioning in quantitative ultrasound imaging. The system 50 is used for an initial or earlier QUS examination and/or for a subsequent or later QUS examination. For initial or earlier, the system 50 provides for entry of indicator of relative position of the transducer 52 to the patient. For later or subsequent QUS examination, the system 50 provides for one or more indicators to assist in placing the QUS ROI on the same anatomy as for the earlier QUS examination.

The system 50 is an ultrasound imager or scanner. In one embodiment, the ultrasound scanner is a medical diagnostic ultrasound imaging system. In alternative embodiments, the ultrasound imager is a personal computer, workstation, PACS station, or other arrangement at a same location or distributed over a network for real-time or post acquisition imaging.

The system 50 implements the method of FIG. 1, the method of FIG. 4, or other methods. The system 50 includes a transmit beamformer 51, a transducer 52, a receive beamformer 53, an image processor 54, a display 55, and a user input 57. Additional, different or fewer components may be provided. For example, a spatial filter, a scan converter, a mapping processor for setting dynamic range, and/or an amplifier for application of gain are provided. As another example, a user input is not provided.

The transmit beamformer 51 is an ultrasound transmitter, memory, pulser, analog circuit, digital circuit, or combinations thereof. The transmit beamformer 51 is configured to generate waveforms for a plurality of channels with different or relative amplitudes, delays, and/or phasing to focus a resulting beam at one or more depths. The waveforms are generated and applied to a transducer array with any timing or pulse repetition frequency.

The transmit beamformer 51 connects with the transducer 52, such as through a transmit/receive switch. Upon transmission of acoustic waves from the transducer 52 in response to the generated waves, one or more beams are formed during a given transmit event. The beams are for B-mode, quantitative mode (e.g., ARFI or shear wave imaging), or other mode of imaging. Sector, Vector®, linear, or other scan formats may be used. The same region is scanned multiple times for generating a sequence of images or for quantification.

The transducer 52 is a 1-, 1.25-, 1.5-, 1.75- or 2-dimensional array of piezoelectric or capacitive membrane elements. The transducer 52 includes a plurality of elements for transducing between acoustic and electrical energies. For example, the transducer 52 is a one-dimensional PZT array with about 64-256 elements. As another example, the transducer 52 is a transesophageal echocardiography (TEE) array, a volume intracardiac echocardiography (ICE) array, or a trans-thoracic echo (TTE) array.

The transducer 52 is releasably connectable with the transmit beamformer 51 for converting electrical waveforms into acoustic waveforms, and with the receive beamformer 53 for converting acoustic echoes into electrical signals. The transducer 52 transmits the transmit beams where the waveforms have a frequency and are focused at a tissue region or location of interest in the patient. The acoustic waveforms are generated in response to applying the electrical waveforms to the transducer elements. The transducer 52 transmits acoustic energy and receives echoes. The receive signals are generated in response to ultrasound energy (echoes) impinging on the elements of the transducer 52.

The transducer 52 is a hand-held probe for use external to the patient. Alternatively, the transducer 52 is part of a probe for insertion within the patient. The transducer 52 may be positioned at various locations relative to the patient by the user and/or by a robotic arm.

The receive beamformer 53 includes a plurality of channels with amplifiers, delays, and/or phase rotators, and one or more summers. Each channel connects with one or more transducer elements. The receive beamformer 53 applies relative delays, phases, and/or apodization to form one or more receive beams in response to each transmission for detection. Dynamic focusing on receive may be provided. The receive beamformer 53 outputs data representing spatial locations using the received acoustic signals. Relative delays and/or phasing and summation of signals from different elements provide beamformation.

The receive beamformer 53 may include a filter, such as a filter for isolating information at a second harmonic or other frequency band relative to the transmit frequency band. Such information may more likely include desired tissue, contrast agent, and/or flow information. In another embodiment, the receive beamformer 53 includes a memory or buffer and a filter or adder. Two or more receive beams are combined to isolate information at a desired frequency band, such as a second harmonic, cubic fundamental, or another band. The fundamental frequency band may alternatively be used.

For ARFI or shear wave imaging, parallel receive beamformation is used. For tracking displacements, a transmit beam covering the ROI is transmitted. Two or more (e.g., 8, 16, 32, or 64) receive beams distributed evenly or unevenly in the ROI are formed in response to each transmit beam.

The receive beamformer 53 outputs beam summed data representing spatial locations. The beam summed data is in an I/Q or RF format. Ultrasound signals are output.

The image processor 54 detects, such as detecting intensity, from the beamformed samples. Any detection may be used, such as B-mode and/or color flow detection. In one embodiment, a B-mode detector is a general processor, application specific integrated circuit, or field programmable gate array. Log compression may be provided by the B-mode detector so that the dynamic range of the B-mode data corresponds to the dynamic range of the display. The image processor 54 may or may not include a scan converter.

The image processor 54 includes a controller, general processor, application specific integrated circuit, field programmable gate array, graphics processing unit, or other processor to position an ROI and perform quantitative ultrasound imaging based on the ROI. The image processor 54 includes or interacts with a beamformer controller to scan the ROI in the QUS scanning. The image processor 54 is configured by hardware, software, and/or firmware.

The image processor 54 may be configured to locate an ROI in a B-mode FOV based on detected data from the scan in the B-mode. For an earlier or initial QUS examination, the ROI is located manually or automatically, such as based on one or more anatomical landmarks represented in the data from the scan in the B-mode. Other modes of scanning may be used. For subsequent QUS examination, the image processor 54 generates indicators to guide placement of the FOV and/or ROI to be for the same anatomy as the earlier examination.

For initial or earlier QUS examination, the image processor 54 is configured to solicit input of or sense relative position of the transducer 52 to the patient during QUS image generation. The relative position as well as the QUS image and ROI position relative to the image or FOV are stored.

For subsequent QUS examination, the image processor 54 is configured to assist positioning of the ROI over a same anatomy of a patient as in a previous instance of QUS imaging. In a different QUS examination of the same patient, one or more indicators are displayed to help position the FOV in the current instance to include the ROI. The image processor 54 generates any indicator, such as a display of a relative position of a transducer to the patient and/or a display of an indication of similarity based on correlation of an image from the previous instance and an image from the current instance. For the relative position, the image processor 54 generates a display of a pictogram or body model indicating a relative position of a transducer to the patient. For the indication of similarity, the image processor 54 determines and displays a similarity between a FOV or the ROI in the previous instance and in the current instance and/or determines and displays a direction of movement of a transducer align with the same anatomy.

The display 55 is a CRT, LCD, monitor, plasma, projector, printer or other device for displaying an image or sequence of images. Any now known or later developed display 55 may be used. The display 20 displays a B-mode image, a QUS image (e.g., annotation or color modulation on a B-mode image), or other images. The display 20 displays one or more images representing the ROI or tissue characteristic in the ROI.

The display 55 is configured by a display plane memory or image generated by the image processor 54. The display 55 is configured to display a current instance of QUS imaging of the same anatomy based on the position of the ROI. The image from the current instance may be displayed simultaneously with an image from the previous instance with quantities of QUS parameters for the ROI in both images being for the same anatomy.

The user input 57 is a mouse, trackball, touchpad, touch screen, keyboard, buttons, sliders, knobs, and/or other input device. The user input 57 operates with the display 55 to provide a user interface generated by the image processor 54. The user may be solicited to provide one or more indications of relative position of the transducer to the patient. The display 55 may be configured by the user interface to output one or more indications with images.

The image processor 54, and/or the ultrasound system 50 operate pursuant to instructions stored in a memory. The instructions configure the system for performance of the acts of FIG. 1 or FIG. 4. The instructions configure for operation by being loaded into a controller, by causing loading of a table of values (e.g., elasticity imaging sequence), and/or by being executed. The memory is a non-transitory computer readable storage media. The instructions for implementing the processes, methods and/or techniques discussed herein are provided on the computer-readable storage media or memories, such as a cache, buffer, RAM, removable media, hard drive or other computer readable storage media. Computer readable storage media include various types of volatile and nonvolatile storage media. The functions, acts, or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing, and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (We) claim:

1. A method for region of interest (ROI) positioning in quantitative ultrasound imaging with an ultrasound scanner, the method comprising:
displaying an indicator of the ROI for first anatomy of a patient from a previous quantitative ultrasound examination, the indicator being different than a representation of the ROI; and
performing another quantitative ultrasound examination with the ROI for this other quantitative ultrasound examination positioned on the first anatomy of the patient based, in part, on the indicator.

2. The method of claim 1 wherein displaying comprises displaying an image of the first anatomy from the previous quantitative ultrasound examination, the image including a graphic for the ROI for the previous quantitative ultrasound examination.

3. The method of claim 1 wherein displaying comprises displaying as part of the other quantitative ultrasound examination, the indicator representing a transducer position relative to the patient from the previous quantitative ultrasound examination, and the indicator being loaded from memory due to creation during the previous quantitative ultrasound examination.

4. The method of claim 1 wherein displaying comprises displaying a pictogram of a transducer position and one or more body markers.

5. The method of claim 1 wherein displaying comprises displaying a body model of internal structure of a body and a transducer position relative to the body model.

6. The method of claim 1 wherein displaying comprises displaying a degree of similarity between the ROI for the first anatomy of the patient and the ROI for the other quantitative ultrasound examination, and further comprising changing an ultrasound field of view to maximize the degree of similarity.

7. The method of claim 1 wherein displaying comprises displaying the indicator as a direction of movement of a transducer probe.

8. The method of claim 1 wherein displaying comprises displaying information relating a transducer position to the patient and then displaying a refinement based on a measure of similarity.

9. The method of claim 1 further comprising positioning a transducer probe relative to the patient by hand based on the indicator.

10. The method of claim 1 wherein performing comprises performing with the ROI for the first anatomy of the other quantitative ultrasound examination at a location of maximum similarity with the ROI for the first anatomy of the previous quantitative ultrasound examination.

11. The method of claim 1 wherein performing comprises generating a quantitative ultrasound image showing values for a quantitative ultrasound parameter for the first anatomy.

12. The method of claim 1 wherein the previous quantitative ultrasound examination is separated by the other quantitative ultrasound examination by twelve hours or more.

13. A method for region of interest (ROI) positioning in quantitative ultrasound imaging with an ultrasound scanner, the method comprising:
storing a quantitative ultrasound image with a region of interest;
receiving input of a relative position of a transducer probe to the patient, the relative position being for when the quantitative ultrasound image is generated; and
storing the relative position linked with the quantitative ultrasound image.

14. The method of claim 13 wherein receiving input comprises receiving user input on a pictogram or body model.

15. A system for region of interest (ROI) positioning in quantitative ultrasound imaging, the system comprising:
an image processor configured to assist positioning of the ROI over a same anatomy of a patient used in a previous instance of quantitative ultrasound imaging; and
a display configured to display a current instance of quantitative ultrasound imaging of the same anatomy based on the position of the ROI.

16. The system of claim 15 wherein the image processor is configured to display on the display a pictogram or body model indicating a relative position of a transducer to the patient.

17. The system of claim 15 wherein the image processor is configured to determine a similarity between a field of view or the ROI in the previous instance and in the current instance.

18. The system of claim 15 wherein the image processor is configured to display an indicator of a direction of movement of a transducer aligned with the same anatomy.

19. The system of claim 15 wherein the image processor is configured to display a relative position of a transducer to the patient and to display an indication based on correlation of an image from the previous instance and an image from the current instance.

20. The system of claim 15 wherein the display is configured to display an image from the current instance simultaneously with an image from the previous instance with quantities of quantitative ultrasound parameters for the ROI in both images being for the same anatomy.
